(19) United States
(12) Patent Application Publication
(10) Pub. No.: US 2013/0303973 A1
(43) Pub. Date: Nov. 14, 2013

(54) DRUG STORAGE AND DELIVERY DEVICE AND METHOD

(71) Applicant: EUCLID SYSTEMS CORPORATION, Herndon, VA (US)

(72) Inventors: Bruce Dewoolfson, Vienna, VA (US); Michael St. John Luttrell, Dayton, OH (US); Thomas Kent Mundorf, Charlotte, NC (US); Eliot Stuart Lazar, Orchard Park, NY (US)

(73) Assignee: Euclid Systems Corporation, Herndon, VA (US)

(21) Appl. No.: 13/889,799

(22) Filed: May 8, 2013

Related U.S. Application Data
(60) Provisional application No. 61/644,485, filed on May 9, 2012.

(51) Int. Cl. A61F 9/00

(52) U.S. Cl. CPC A61F 9/0008
USPC 604/22

(57) ABSTRACT

A drug delivery device and method is disclosed. The drug delivery device may include a cutting portion configured to form a slit in a portion of eye tissue. The drug device may also include a sliding assembly configured to slide with respect to the cutting portion to deliver a biopharmaceutical device to the portion of eye tissue.

### TECHNICAL FIELD

The present disclosure relates generally to drug delivery devices, and more particularly to drug delivery devices for treatment of ophthalmic diseases.

### BACKGROUND

The treatment of various diseases, for example, ophthalmic diseases, and post-operative conditions often requires administration of drugs to the ocular tissues. One approach for achieving localized drug delivery involves the injection of a drug directly under the conjunctiva or tenon's capsule, intra-camerally or intra-vitreally. In other instances, medications are applied topically to the eye, for example, by using drops or ointments. The topical formulation is administered by the patient or caregiver using an eye dropper or dispenser.

In addition to injection and topical drug administration, in some instances ocular inserts may be provided to continuously deliver active agents to the eye. Some inserts disperse the drug and require removal of a carrier of the drug once the drug has been delivered, while other inserts erode in place, and thus do not require removal after drug delivery.

U.S. Pat. No. 7,044,945 to Sand (the '945 patent) describes various methods and apparatuses for providing therapeutic agents to prevent epithelial apoptosis and/or modulation of a wound-healing process at selected points, specifically stromal remodeling. The '945 patent discloses flexible capsules or wafers, or other membrane devices that can be placed in the conjunctival sac. The membrane device is left in the conjunctival sac for about five minutes, or until the drug or reagent sufficiently penetrates the cornea. The '945 patent further discloses administration by subconjunctival injection using a syringe to penetrate and deliver reagent to the subconjunctva.

### SUMMARY

In one aspect, a drug delivery device is disclosed. The drug delivery device may include a cutting portion configured to form a slit in a portion of eye tissue. The drug delivery device may also include a sliding assembly configured to slide with respect to the cutting portion to deliver a biopharmaceutical device to the portion of eye tissue.

In another aspect, a drug delivery device is disclosed. The drug delivery device may include a cutting portion configured to form a slit in a portion of eye tissue, and a biopharmaceutical device for sustained delivery of ophthalmic biopharmaceuticals. The drug delivery device may further include a sliding assembly configured to deliver the biopharmaceutical device to the portion of eye tissue.

In yet another aspect, a method of treating an ophthalmic disease is disclosed. The method may include inserting a cutting portion of a drug delivery device into a portion of eye tissue, and dispensing a biopharmaceutical device from the drug delivery device into a slit in the portion of the eye tissue formed by the cutting portion.

### BRIEF DESCRIPTION OF THE DRAWINGS

FIG. 1 is an isometric view of a drug delivery device;

FIG. 2 is a top view of the drug delivery device of FIG. 1;

FIG. 3 is a view along lines 3-3 of FIG. 2;

FIG. 4 is a magnified view of a portion IV of the drug delivery device of FIG. 3;

FIG, 5 is an exploded view of the drug delivery device of FIG. 1;

FIG. 6 is a flow chart showing the steps to assemble the drug delivery device of FIGS. 1-5; and

FIG. 7 is a flow chart showing the steps to deliver a drug using the drug delivery device of FIGS. 1-5.

### DETAILED DESCRIPTION

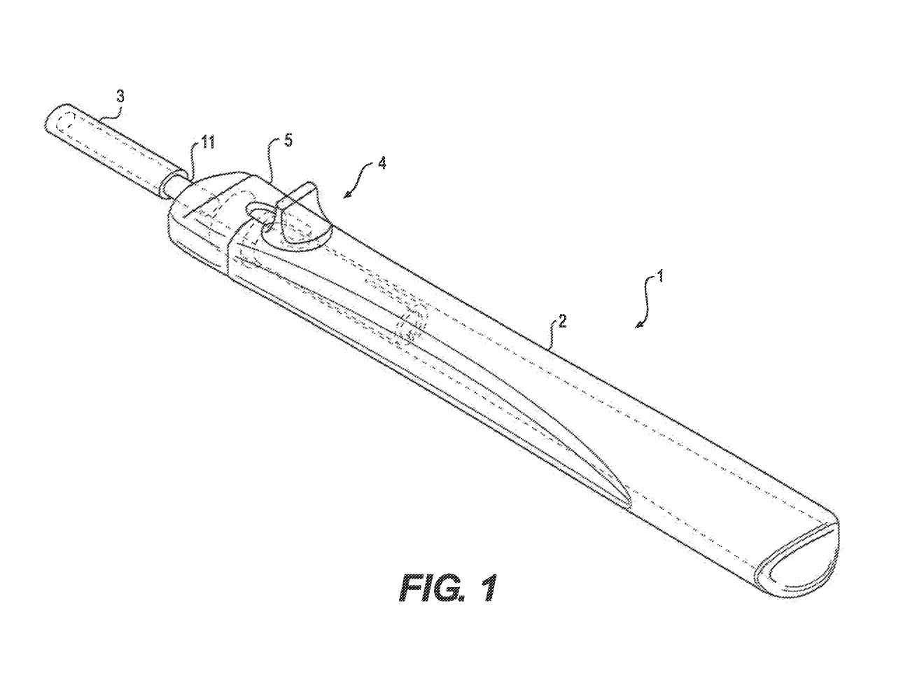

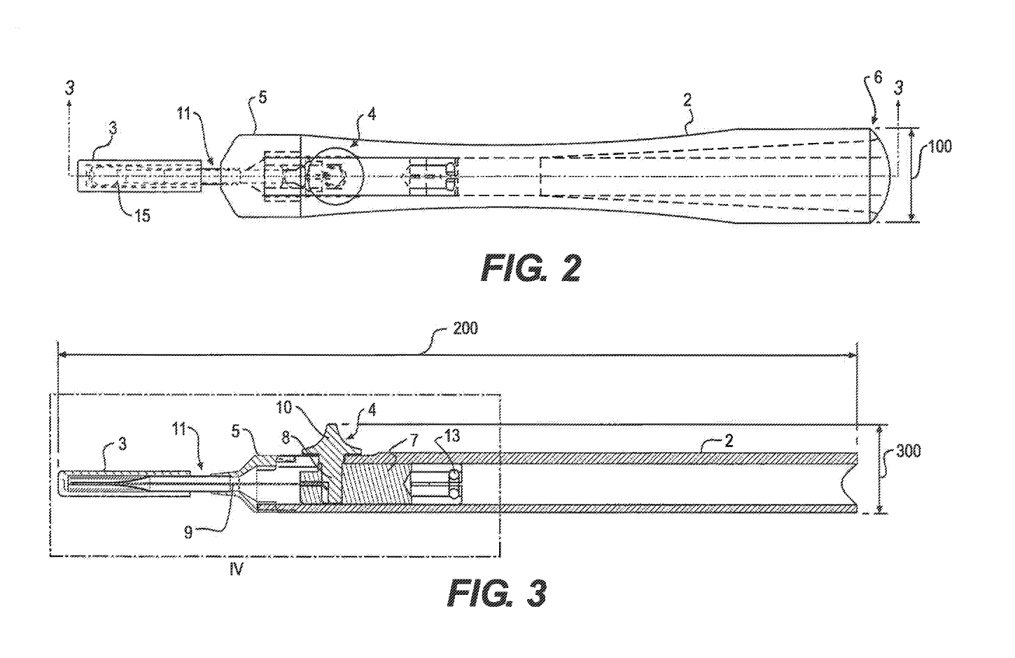

FIG. 1 illustrates an isometric view of a drug delivery device 1 capable of storing and delivering a drug. The drug delivery device 1 includes a handle 2, a holder portion 5 connected to the handle 2, and a cutting portion 11 connected to the holder portion 5. The handle 2 and holder portion 5 may be referred to herein as a “handle assembly.” In some embodiments, the drug delivery device may include a removable cap 3 for covering at least part of the cutting portion 11. A user, for example a doctor, may selectively place the cap 3 over the cutting portion 11 or remove the cap 3 from the cutting portion 11. A knob 4 extends from the handle 2 and is slidable between the handle 2 and the holding portion 5, which is explained in more detail below. The dashed lines in FIG. 1, as well as the dashed lines in FIGS. 2 and 5, indicate internal elements, for example drug delivery device components or various internal walls, that cannot be seen by a person viewing the assembled drug delivery device 1.

In some embodiments, at least one of the handle 2, the cap 3, and the knob 4 may be injection molded plastic. In one example, the handle 2, cap 3, and knob 4 are all injection molded plastic. The handle 2 may be a single molded piece or, alternatively, the handle 2 may be a plurality of molded pieces assemble together to form the handle 2. The cutting portion 11, explained in more detail below, may be steel, for example, stainless steel hypodermic needle tubing.

FIG. 2 is a top view of the drug delivery device 1 of FIG. 1. As shown in FIG. 2, a back end 6 of the handle 2 has a width 100. In some embodiments, the width 100 may be about 1.0-2.0 cm (about 0.4-0.8 inches), or about 1.524 cm (about 0.600 inches). The handle 2 shown in FIG. 2 includes a curvature extending between the back end 6 and the holding portion 5, the curvature capable of accommodating a grip of a user of the drug delivery device 1. A wafer 15, to be described in more detail below, is shown by dashed lines within the cutting portion 11 and the cap 3.

FIG. 3 is a view of the drug delivery device along lines 3-3 of FIG, 2. As shown in FIG. 3, a slidable element 7 (herein referred to as a “slider”) is disposed within at least a portion of the handle 2. The slider 7 may also be disposed within at least a portion of the holder portion 5. In some instances, a fitting element 13, for example an o-ring, may be disposed within the slider 7. As shown in FIG. 3, the fitting element 13 is disposed within a portion of the slider 7 that is disposed within the handle 2. The slider 7 may include a step 47 at one end of the slider 7, for example, at an end of the slider closest to the holder portion 5.

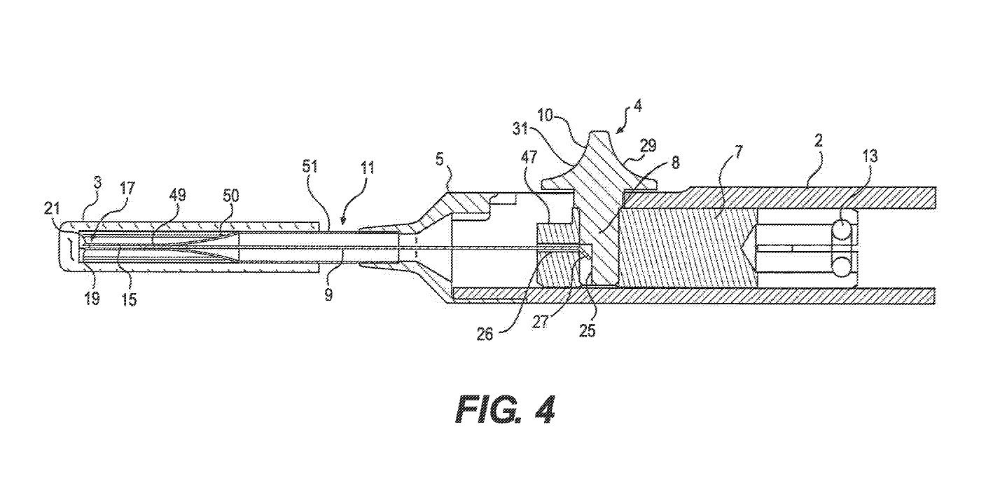

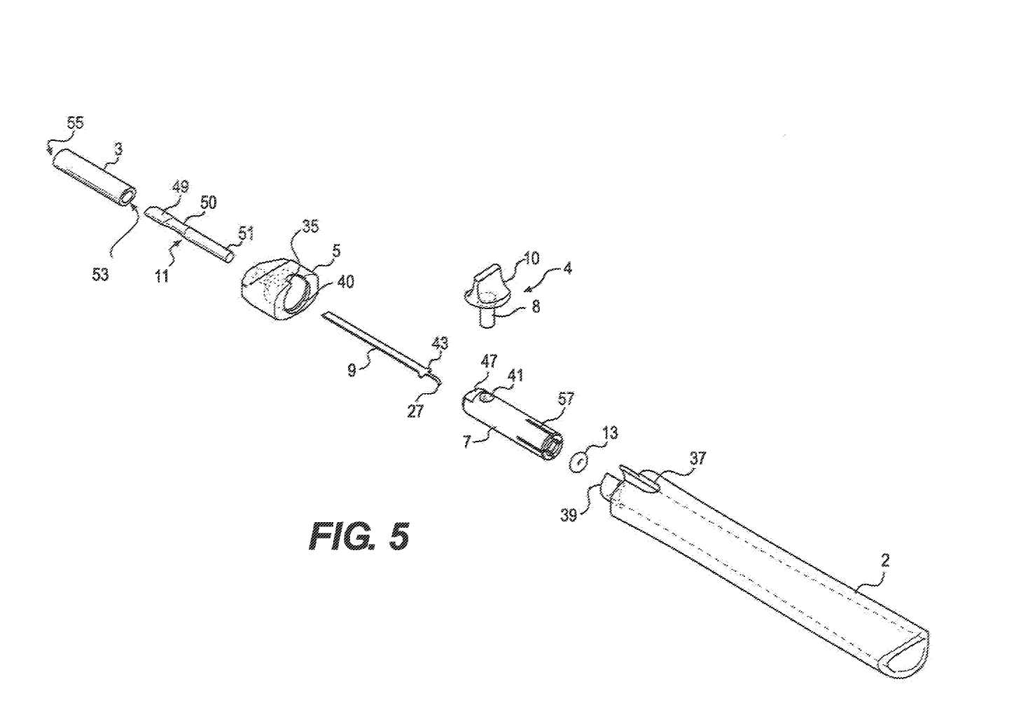

As shown in FIG. 3, the knob 4 includes a contact portion 10, and a stem 8 extending into an interior of the handle 2. The stem 8 is disposed in a cavity 41 of the slider 7 (FIG. 5). The holder portion 5 is connected to the handle 2, as mentioned above, near the knob 4, for example, adjacent the knob 4. The cutting portion 11, which is connected to the holder portion 5, may have a pushing element 9 disposed therein for pushing the wafer 15 (FIGS. 2 and 4), as described in more detail below. The pushing element 9 may also be referred to herein, for example, as a bar, a rod, or the like. As shown in FIG. 3, the pushing element 9 may extend within at least part of the cutting portion 11, the holder portion 5, and the slider 7. The knob 4, slider 7, and pushing element 9 may collectively be referred to herein as a sliding assembly.

As shown in FIG. 3, the drug delivery device has a length 200. In some embodiments, the length 200 may be about 10-15 cm (about 3.94-5.91 inches), or about 13.56 cm (about 5.34 inches). The drug delivery device 1 also has a height 300. In some embodiments, the height 300 may be about 1.27-1.52 cm (about 0.50-0.60 inches), or about 1.37 cm (0.54 inches).

In FIG. 3, the knob 4 is shown in a first (also referred to herein as a “retracted”) position, that is, a position in which the pushing element 9 is retracted such that a wafer 15 (FIG. 4) is within the cutting portion 11. As described in more detail below, the knob 4 may be moved, for example by a user, from the retracted position shown in FIG. 3 to a second (also referred to herein as an “advanced”) position (not shown). In the advanced position, the wafer 15 is dispensed from the drug delivery device 1.

The wafer 15, which can be a collagen wafer, may be referred to herein as, for example, an “ophthalmic biopharmaceutical device,” an “intraocular wafer,” an “ocular insert,” a “medicinal wafer,” a “device for sustained delivery of ophthalmic biopharmaceuticals,” or a “biodegradable wafer” such as a “biodegradable collagen wafer.” As described in more detail below, the wafer 15 may be frozen, at least at the time the wafer 15 is disposed within the drug delivery device 1. In some embodiments, when the wafer 15 is frozen, the wafer 15 may be generally flat, that is, in a frozen state the wafer 15 may not be curved or bowed.

The drug delivery device 1 is configured to store and dispense one wafer 15 before the entire drug delivery device 1 is thrown away. In some instances, as shown in FIG. 2 the wafer 15 is substantially rectangular, and the corners of the wafer 15 may be rounded. When the wafer 15 has a substantially rectangular shape, the wafer 15 may have a length of about 4 mm (0.16 inches) and a width of about 3 mm (about 0.12 inches). Additionally, the wafer 15 may have a thickness substantially the same as a thickness between a first tip 19 and a second tip 21 of the cutting portion 11, described in more detail below. In some embodiments, the thickness of the wafer to fit within the cutting portion 11 can be about 0.127 mm (about 0.005 inches). Depending on the application and the patient, however, either thicker or thinner wafers 15 may be used. Additionally, the shape of the wafer 15 is not limited to a substantially rectangular shape. In some embodiments, the wafer 15 may have either a circular or ovular shape. Additionally, the wafer 15 may be a carrier for one of a variety of drugs or medicines, for example, one or more prostaglandins. Furthermore, the wafer 15 may be color coded such that the color of the wafer 15 indicates the type of drug carried by the wafer 15.

FIG. 4 is a magnified view of a portion IV of the drug delivery device of FIG. 3. As shown in FIG. 4, the knob 4, specifically the stem 8 of the knob 4, can include a notch 25. In some instances, the notch 25 accommodates an end 27 of the pushing element 9. As shown in FIG. 4, the end 27 of the pushing element 9 may be bent to fit within the notch 25. The pushing element 9 may extend from the notch 25 through a passage 26 of the slider, through the holder portion 5, and through an interior of the cutting portion 11. In some embodiments, the pushing element 9 may extend through a middle of the cutting portion.

The cutting portion 11 may include a plurality of sections, for example a first section 51, a second section 50, and a third section 49. In some instances, the first section 51 may be a tubular section (FIG. 5) connected to the holder portion 5. The second section 50 may be tapered or curved from the first section 51, such that the second section 50 narrows the cutting portion 11 towards the third section 49. The second section 50 may have a transitional shape, that is, a portion of the second section 50 adjacent the first section 51 may be tubular, while a portion of the second section 50 adjacent the third section 49 may be substantially flat (FIG. 5).

As shown in FIG. 4, the third section 49 may include a tip portion 17, the tip portion 17 having the first tip 19 and the second tip 21. The first and second tips 19 and 21, respectively, may be spaced from each other so that the wafer 15 may be stored therebetween. As noted above, the distance or spacing between the first tip 19 and the second tip 21 may be substantially the same as the thickness of the wafer 15 stored in the tip portion 17. As shown in FIG. 4, the wafer 15 may be stored within the cutting portion 11 adjacent the first tip 19 and the second tip 21. When the cap 3 is in place over at least a portion of the cutting portion 11, the cap 3 may cover part of the first section 51, and all of the second and third sections 50 and 49, respectively. Additionally, while the cutting portion 11 of FIG. 4 is shown as a single, unitary element, the plurality of sections of the cutting portion 11 may be made either integrally or separately and then assembled.

The contact portion 10 of the knob 4 may extend from the stem 8 exterior to the handle 2. As shown in the drug delivery device 1 of FIG. 4, the contact portion 10 includes a first contour 29 and a second contour 31. The contours 29 and 31 may allow a user to use a thumb or finger, for example, to move the knob 4, which, as described in more detail below, can dispense the wafer 15. In other embodiments, the contact portion 10 may include any number of contour portions, for example, one contour portion, such as contour portion 29, or three or more contour portions.

The various elements of the drug delivery device 1 may be held together by one or a combination of a variety of fixation techniques. For example, the stem 8 may be press-fit within the cavity 41 of the slider 7, and/or an adhesive may be used to fix the stem 8 within the cavity 41. The pushing element 9 may be fixed to the slider 7, in a similar fashion, by a press-fit, adhesive, and/or other fixation technique. The holder portion 5 may be snap-fit onto the handle 2, specifically onto the insertion portion 39 of the handle, and the cutting portion 11 may be molder, for example insert molded, onto the holder portion 5, The cap 3 may fit closely over the cutting portion 11 to operate as a protective cover. The slider 7 is configured to slide within an interior of the handle 2, as described in more detail below, by the inherent lubricity of the plastic of the slider 7 and an inner wall of the handle 2. Therefore, additional lubricating means, such as liquid lubricant, is not required to facilitate sliding of the slider 7 within the handle 2.

FIG. 5 illustrates an exploded view of the drug delivery device 1 of FIG. 1. As shown in FIG. 5, the handle 2 includes a first cutout 37 capable of receiving the knob 4, specifically the stem 8 of the knob 4. The handle 2 further includes an insertion portion 39 capable of being inserted into a receiving portion 40 of the holder portion 5. The holder portion 5 may include a second cutout 35 capable of receiving the knob 4, specifically the stem 8 of the knob 4. As described in more detail below, the knob 4 may be slidable within the first and second cutout portions 37 and 35, respectively. The length of the cutout portions 37 and 35 can be controlled to regulate the extension or stroke of the pushing element 9, which, as described below, controls delivery of the wafer 15 into a portion of the eye.

As shown in FIG. 5, the slider 7 may include at least one slot 57. In some embodiments, the slider 7 includes a plurality of slots 57, for example four slots 57, configured to receive the fitting element 13. The fitting element 13 can exert a pushing or spring-like force on the slider 7, which causes the portion of the slider having the slots 57 to expand or flare outward. With the fitting element 13, such as an o-ring, in place in the slots 57, the slider 7 can fit snuggly within the open internal space of the handle 2 (FIGS. 1 and 2). As shown in FIGS. 1, 2 and 5, the open internal space within the handle can taper outwardly. Thus, providing the fitting element 13 within the slider 7 as shown secures the slider 7 in place within the handle 2.

As shown in FIG. 5, the pushing element 9 may be an elongated bar or rod, including a widened portion 43. When the drug delivery device 1 is assembled, the widened portion 43 may be disposed within the slider 7, along with the end 27 (FIG. 2). Additionally, the cap 3 may include a first end 53 and a second end 55, the second end 55 being a distalmost end of the drug delivery device 1.

### INDUSTRIAL APPLICABILITY

The described device may be used by, for example, surgeons treating a variety of ocular diseases or conditions, such as glaucoma, a cataract, or other diseases. The device may also be used to insert a biodegradable wafer carrying a medicine, for example a prostaglandin, into another part of a body requiring sustained delivery of drugs, such as biopharmaceuticals.

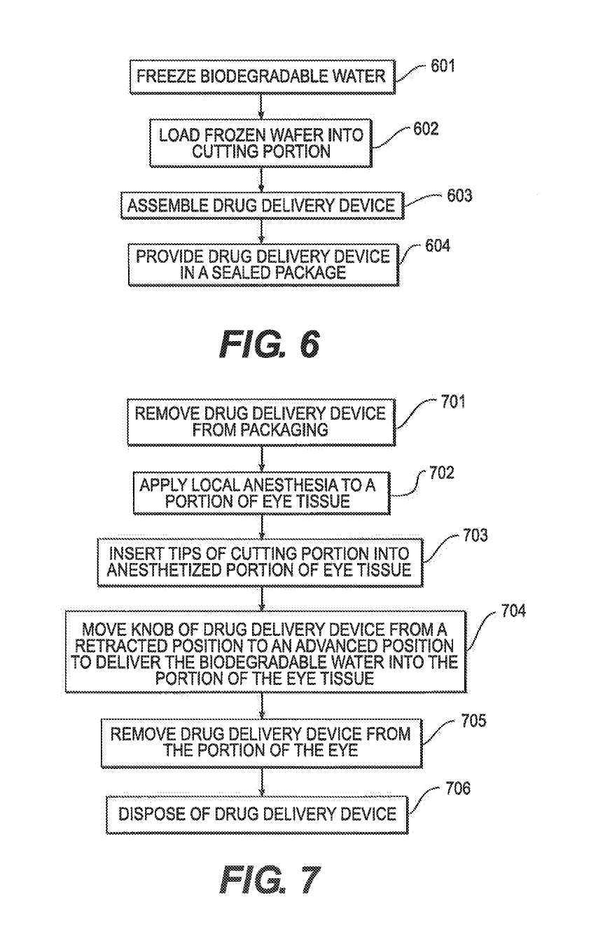

The following operation will be directed to the assembly and use of the drug delivery device 1 of FIGS. 1-5. FIG. 6 is a flow chart showing the steps to assemble the drug delivery device 1 of FIGS. 1-5. In step 601, the wafer 15 is frozen and/or dried. Freezing the wafer 15 may be accomplished by placing the wafer in a freezer (not shown) having the capacity to freeze the drug of the wafer 15. In step 602, the wafer 15 is loaded into the cutting portion 11 of the drug delivery device 1. Specifically, the wafer 15 is loaded into the third section 49 of the cutting portion 11, adjacent the top portion 17, as shown in FIG. 4, The wafer 15 may be manually or automatically loaded (also referred to herein as “preloaded”) into the cutting portion 11.

In step 603 of FIG, 6, the components of the drug delivery device 1 (FIG. 5) are assembled via commonly known fixation techniques as described above. Thus, after step 603, the drug delivery device 1 is fully assembled with the frozen wafer 15 stored therein. In step 603, the components of the drug delivery device 1 may be assembled either manually or automatically. In step 604, the assembled drug delivery device 1 can be provided in sealed, ready-to-use packaging for storage and/or transportation. In some instances, the wafer 15 may be maintained in a frozen state after being placed in the drug delivery device 1. To do so, the entire drug delivery device 1, including the wafer 15 stored therein, may be stored at a sufficiently low temperature, for example, in a freezer, for a period of time until the drug delivery device is ready to be used as described below.

As noted above, the drug delivery device 1 can be used to deliver drugs in the form of biodegradable wafers 15 into the tissue of the eye. FIG. 7 is a flow chart showing the steps to deliver a drug using the drug delivery device of FIGS. 1-5. As used herein, the term “user” may refer to any person, for example, a doctor, who may use the drug delivery device 1. After identifying a need to treat a condition, such as glaucoma or a cataract, in step 701 a user or other person may remove the drug delivery device 1 from the packaging. In a step 702, the user or other person may apply local anesthesia to a portion of the eye (not shown). Step 702 is optional, and steps 701 and 702 may be performed in reverse order, that is, the portion of the eye may be anesthetized prior to removal of the drug delivery device 1 from the packaging.

In step 703, the user inserts the cutting portion, specifically, the tip portion 17, into the anesthetized portion of the eye tissue. Insertion of the tip portion results in an incision in the portion of the eye tissue, which is facilitated by the sharp first and second tips 19 and 21, respectively, of the tip portion 17. The incision, also referred to as a cut or slit, in the portion of the eye tissue forms a pocket capable of receiving and retaining the wafer 15. The incision may be a slit having a length of about 3.5-4.0 mm (0.14-0.16 inches). In some embodiments, the pocket is formed in or leading to the subconjunctival tissue, that is, the space or tissue behind the conjunctival tissue where the eye can rotate. While the tip portion 17 is within the portion of the eye tissue, in step 704 the user maintains the position of the drug delivery device 1, and moves the knob 4 from the retracted position to the advanced position to dispense the wafer 15 from the drug delivery device 1 and into the pocket in the portion of the eye tissue. Thus, in step 704 the wafer 15 is placed in the subconjunctival tissue, or subconjunctival area, of the eye, for the sustained delivery of a drug to the eye. Due to the connectedness of the components of the drug delivery device, that is, the knob 4, the slider 7, the pushing element 9, the cutting portion 11, and the wafer 15, moving the knob 4 from the retracted position to the advanced position pushes the wafer 15 out of the cutting portion 11 and into the pocket in the portion of the eye tissue.

In step 705, after the wafer 15 is disposed within the pocket in the portion of the eye tissue, the user removes the drug delivery device 1 from the eye. In step 706, the user or another person disposes of the drug delivery device 1. As a final, optional step, a user may directly apply a protein, for example, decorin, directly to the site of the wound, that is, the location where the incision was made in the eye tissue. The application of decorin in this manner may expedite the healing process. In the absence of decorin, however, there may not be a need for a suture due to the small size of the wound, that is, the slit, and the self-healing nature of the eye tissue.

Topical administration of a medication for treatment of an ocular condition or disease may result in drainage of the medication from the ocular surface into the lacrimal system through an opening in the eyelid called the punctum. Furthermore, the medication may be diluted by tears secreted by the lacrimal gland. Furthermore, topical formulations generally require application by a patient or caregiver of the proper dose at the proper time. Thus, topical treatments do not provide a continuous, prolonged delivery of medication and the exact dosage achieved at the target tissue is unpredictable. Intermittent administration may also be problematic because there is likely to be an initial overdosage followed by a rapid decrease in concentration due to dilution and lacrimal drainage to ineffective levels. Even with injection delivery systems, there is typically the inconvenient need to return to a doctor approximately every four months to receive additional, often uncomfortable, injections. Additionally, separate instruments must often be used (1) to make an incision in a portion of body tissue, and (2) to deliver a drug or other device to the body through the incision.

The drug delivery device 1 described herein employs a unitary design, that is, a single device that can make an incision and dispense and place a medicine at a desired location. A single drug delivery device 1 shown in FIGS. 1-5 may conveniently form a slit in a portion of eye tissue via the cutting portion 11, and dispense and position a wafer 15 at a desired location, for example, within the subconjunctival tissue of the eye, for the sustained treatment of a medical condition or disease. Furthermore, as noted above, the entire device 1 may be a one-use device, such that a user may dispose of the entire device 1 after treating a patient. The device 1 is inexpensive to manufacture, and thus can be disposed of after a single use. For example, a user may treat one eye of a patient, dispose of the device 1, and use another device 1 to treat another eye of the same patient, or an eye of a different patient.

Unlike injection devices, the drug delivery device 1 described herein may only require a patient to visit a doctor once every six months for retreatment. Due to the small incision size made by the drug delivery device 1, a patient receiving treatment does not likely experience substantial discomfort or pain during or after treatment. Additionally, a patient can receive treatment with the drug delivery device 1 in, for example, an ambulatory surgical center (“ASC”), or another similar location. Because an ASC is an outpatient, or same-day, facility, a patient being treated with the above-described device is not burdened by having to commit time or funds to an overnight hospital stay.

Regarding the freezing of the wafer 15 prior to loading the wafer 15 into the device 1, doing so may provide stiffness and structural rigidity and integrity to ease insertion of the wafer into the device 1 and/or into the portion of the eye during treatment. If the wafer 15 is frozen prior to being dispensed into the body of a patient, the wafers will eventually warm due to heat from the patient's body. Providing a wafer 15 as an imperfect rectangle, that is, a rectangle having rounded corners, may facilitate insertion into and retention within the subconjunctival tissue of the eye. This, in turn, may facilitate the sustained delivery of the drug carried by the wafer 15 and treatment of a disease. Furthermore, as noted above, due to the small size of the slit made in the portion of the eye tissue and the self-healing nature of the eye, it is not necessary to provide a surgical suture to the wound.

As an alternative, although the above-described drug delivery device 1 is configured to store and dispense one wafer 15, alternate embodiments of the drug delivery device 1 may store and dispense a plurality of wafers 15. Additionally, as another alternative, a drug delivery device may be provided having a biopharmaceutical device, such as a medicinal wafer, stored therein. A user may depress a pushing element of the device with a thumb or forefinger in order to dispense the wafer into a portion of eye tissue. Such a drug delivery device may include at least one sharp edge at a dispensing end of the device to form an incision into the portion of eye tissue, where the wafer can be dispensed from adjacent the at least one sharp edge. This alternative drug delivery device may be a unitary plastic device, having at least one contour for a user to place, for example, a thumb. Additionally, such a drug delivery device may be inexpensive to manufacture and disposable after a single treatment or use.

It will be apparent to those skilled in the art that various additional modifications and variations can be made to the disclosed collagen wafer device and method. Other embodiments will be apparent to those skilled in the art from consideration of the specification and practice of the disclosed system and method. It is intended that the specification and examples be considered as exemplary only, with a true scope being indicated by the following claims and their equivalents.

What is claimed is:

1. A drug delivery device comprising:
a cutting portion configured to form a slit in a portion of eye tissue; and
a sliding assembly configured to slide with respect to the cutting portion to deliver a biopharmaceutical device to the portion of eye tissue.

2. The drug delivery device of claim 1, wherein the sliding assembly is slidable within a handle.

3. The drug delivery device of claim 1, wherein the sliding assembly comprises:
a knob configured to move from a first position to a second position to deliver the biopharmaceutical device.

4. The drug delivery device of claim 1, wherein the biopharmaceutical device is a collagen wafer.

5. The drug delivery device of claim 1, wherein the sliding assembly comprises:
a pushing element configured to contact the biopharmaceutical device during delivery of the biopharmaceutical device to the portion of eye tissue.

6. The drug delivery device of claim 1, wherein the cutting portion comprises:
at least one tip configured to form the slit,

7. The drug delivery device of claim 6, wherein the at least one tip comprises a plurality of tips disposed at an end of the cutting portion.

8. The drug delivery device of claim 6, wherein the biopharmaceutical device is held within the cutting portion adjacent the at least one tip prior to delivery of the biopharmaceutical device.

9. The drug delivery device of claim 1, further comprising;
a handle assembly configured to retain the sliding assembly and the cutting portion.

10. A drug delivery device comprising:
a cutting portion configured to form a slit in a portion of eye tissue;
a biopharmaceutical device for sustained delivery of ophthalmic biopharmaceuticals; and
a sliding assembly configured to deliver the biopharmaceutical device to the portion of eye tissue.

11. The drug delivery device of claim 10, wherein the sliding assembly comprises:
a slider configured to move in at least one direction within a portion of the drug delivery device; and
a pushing element configured to contact the biopharmaceutical device during delivery of the biopharmaceutical device to the portion of eye tissue.

12. The drug delivery device of claim 10, wherein the biopharmaceutical device is a wafer disposed within the sliding assembly.

13. The drug delivery device of claim 10, wherein the biopharmaceutical device is a frozen wafer.

14. The drug delivery device of claim 10, wherein the sliding assembly is configured to move with respect to a handle assembly of the drug delivery device.

15. A method of treating an ophthalmic disease comprising:
inserting a cutting portion of a drug delivery device into a portion of eye tissue; and
dispensing a biopharmaceutical device from the drug delivery device into a slit in the portion of the eye tissue formed by the cutting portion.

16. The method of claim 15, wherein the dispensing is performed after the inserting without removing the cutting portion of the drug delivery device from the portion of the eye tissue.

17. The method of claim 15, wherein the dispensing is performed manually.

18. The method of claim 15, wherein the biopharmaceutical device is a collagen wafer.

19. The method of claim 15, wherein the dispensing places the biopharmaceutical device into subconjunctival tissue of the eye.

20. The method of claim 15, further comprising:
disposing of the drug delivery device after the dispensing.
